(19) United States
(12) Patent Application Publication
(10) Pub. No.: US 2010/0119578 A1
(43) Pub. Date: May 13, 2010

(54) EXTRACELLULAR MATRIX MODULATING COATINGS FOR MEDICAL DEVICES

(75) Inventors: John Thao To, Newark, CA (US); Charles M. Blaha, San Francisco, CA (US); Loc Xuan Phan, San Jose, CA (US)

Correspondence Address: VISTA IP LAW GROUP LLP, 12930 Saratoga Avenue, Suite D-2, Saratoga, CA, 95070, US

(73) Assignee: SPECIALIZED VASCULAR TECHNOLOGIES, INC., Fremont, CA (US)

(21) Appl. No.: 12/613,477

(22) Filed: Nov. 5, 2009

Related U.S. Application Data
(60) Provisional application No. 61/112,667, filed on Nov. 7, 2008.

(51) Int. Cl. A61F 2/82; A61K 31/70; A61K 31/47; A61K 31/4965

(52) U.S. Cl. 424/423; 514/62; 514/311; 514/255.01; 623/1.46

(57) ABSTRACT

A unique method and coatings are provided to promote/allow early stage tissue encapsulation/endothelization of medical devices while effectively controlling excessive tissue buildup by eluting antiproliferative therapeutic agent within a body of a patient. The method involves using a therapeutic agent that suppresses excessive extracellular matrix proliferation and allows/promotes thin tissue healing/encapsulation/endothelization of the device. Optimized timing of onset of elution to match onset of excessive extracellular matrix proliferation for maximum effectiveness is achieved by delay barrier with biochemical switch.

### RELATED APPLICATION DATA

The present application claims the benefit under 35 U.S.C. §119 to U.S. provisional patent application Ser. No. 61/112,667, filed Nov. 7, 2008. The foregoing application is hereby incorporated by reference into the present application in its entirety.

### BACKGROUND OF THE INVENTION

1. Field of the Invention

The present invention relates to devices and methods for preventing reclosure of a vascular vessel after a surgical procedure therein. More specifically, when the surgical procedure is the implantation of a stent in a coronary vessel, the invention relates to devices and methods for promoting the body's acceptance of the stent, with or without therapeutic agent elution, by controlling immune responses. Most specifically, the invention relates to stents that operate, at least in part, by influencing the development of the extracellular matrix (ECM).

2. Description of the Related Art

Coronary heart disease is a major cause of death in the western world. Most cases of coronary disease involve atherosclerosis in which the heart's vessels become clogged with plaque and fatty deposits to constrict the flow of blood. Modern approaches to restore blood flow and counteract the development of the disease include percutaneous transluminal coronary angioplasty (PTCA) and coronary artery bypass graft (CABG). PTCA is preferably because it is less invasive. However, PTCA alone is frequently unsuccessful in the long-term due to post-angioplasty reclosure of the vessel. Accordingly, common approaches implant long-lasting prosthetics, such as stents, to hold the vessel open after the balloon-tipped catheter used in a PTCA procedure is removed. Modern stents include therapeutic agents to address the reclosure problem from both a chemical and a mechanical perspective. Despite the resources that have been devoted to address this problem of post-angioplasty vessel reclosure the current stents are less than perfect and the need for a better solution still exists. (see U.S. Pat. No. 7,223,286)

Presently, two therapeutic agent eluting stents (DES) on the market that have s demonstrated tremendous effectiveness in minimizing in-stent restenosis are the Cypher® (rapamycin) and Taxus® (paclitaxel) stents. However, both of these stents suffer from the risk of late stage thrombosis (LST) which is a safety problem. So they may be effective but not safe.

It is well documented that the problem with the existing therapeutic agent eluting stents (DES) is that they prevent the struts from being completely healed over by endothelium and thus can cause thrombosis in the long term or late stage thrombosis Since stents are foreign body materials, they cause thrombus formation as the body reacts to their exposure in the blood stream. This can lead to rapid occlusion of a blood vessel causing severe complications to the patient as a result. Antiplatelet therapeutic agent therapy (i.e. using chlopidogrel) is a common way to prevent thrombosis from occurring. When bare metal stents (BMS) are used, oral administration of a systematic antiplatelet therapeutic agent is typically prescribed for a month after implantation. However, in DES an antiplatelet therapeutic agent is prescribed indefinitely and can pose a danger to a patient who unexpectedly has to go into surgery. The uncontrollable bleeding encouraged by antiplatelet therapeutic agents is a serious risk factor that may even cause a patient to die. Additionally, with DES and orally administered therapeutic agents, if a patient forgets to take the therapeutic agent or cannot afford it, the patient may suffer an ischemic attack or death from stent thrombosis.

Other attempts to reduce the risk of LST utilize different methods and mechanisms for releasing the restenosis-preventing therapeutic agents. These include: (i) using different materials [fluoropolymer, phosphorylcholine (PC), polylactic acid (PLA), polyglycolic acid (PGA) combined with PLA, hydroxyapatite (HA), etc. . . . ] as matrices to contain the therapeutic agent, (ii) varying the geometric features of the surface (porous surfaces, micro-wells, micro-holes), (iii) using different types of therapeutic agents (Everolimus, Biolimus, Zotarolimus, Tacrolimus), (iv) changing therapeutic agent release rate profiles, and/or (v) using different type of coatings (PC, collagen) on the stent surfaces to encourage endothelization. None of these approaches have proven effective in eliminating LST while maintaining the high effectiveness in preventing restenosis as Cypher™ and Taxus™. The present invention emphasizes stent coating geometry (i.e. aligned) and therapeutic agent release rate profile (extended delayed onset followed by rapid burst release).

References in the art refer to a delay coating in the context of a coating that protects and suppresses elution of the therapeutic agent during the stent implantation phase (see FIG. 2). It is well known in the art to prevent elution of the therapeutic agent from the stent while the stent is being delivered and positioned within the body. The objective is to avoid systematic loss of the therapeutic agent before the stent reaches its target location. However, once the stent is in place, the reference art considers the timing appropriate to begin therapeutic agent elution for a localized effect. The therapeutic agent eluting stent of the present invention differs from the approaches of the reference art because the coating survives after the placement of the stent in its target position. In the present invention, substantial therapeutic agent elution does not begin immediately upon stent placement. Rather, the delay coating is used to restrain therapeutic agent elution both during and after stent placement. According to the present invention, even after the stent is properly situated, the delay coating should continue to prohibit the distribution of the antirestenosis therapeutic agent for 20-60 days in order to allow sufficient time for beneficial healing and tissue encapsulation of the foreign material and struts that comprise the stent. Nonetheless, in the delayed onset coating of the present invention the initial elution rate of the therapeutic agent immediately after the stent is implanted need not be zero. Rather, a coating may be considered to be a suitable “delayed onset” coating so long as the initial amount and/or rate of elution is very low compared to a later amount and/or rate.

Physicians typically prescribe antiplatelet therapeutic agent therapy for the patient with a bare metal stent (BMS) only for 30 days because neointima tend to cover the stent strut in that period and so mask the foreign body from blood (see FIG. 1). Since BMS do not elute any therapeutic agents, including restenosis-inhibiting therapeutic agents that prevent neointima from developing, the stent struts get covered unlike the situation in most conventional DES. It is well documented that thrombosis (LST) rarely occurs in BMS in the late stage (past 6 months). Neointima helps to smooth out interruptions in the vascular lumen caused by the stent struts which improves hemodynamics. Surface smoothness minimizes stagnate pockets of flow or low velocity/low shear blood flow and this reduces the risk of thrombus formation. The risk with BMS is more likely to be uncontrolled restenosis rather than thrombosis because no long-term antirestenosis therapeutic agents are administered locally to bring restenosis to a halt.

Recent research efforts have emphasized the role of the polymer matrix, in which a therapeutic agent is embedded or coated, in causing restenosis and thrombosis. Consequently, product development has focused on eliminating or modifying the composition of the polymer or substituting new therapeutic agents (i.e. see U.S. Pat. No. 7,279,175).

Based on the assumption that the foreign materials in traditional polymer stent coatings are responsible for producing an immune reaction and late stent thrombosis (LST), the company MIV Therapeutics, Inc. has focused on the design of a polymer-free, bioabsorbable hydroxyapatite coating (see SISM Research & Investment Services article of Apr. 26, 2007 re: MIV Therapeutics, Inc.). Overemphasizing the importance of the chemical composition of the polymer material teaches away from one of the present invention's main solutions (among several solutions) to the problem of an uncontrolled immune response. In addition to teaching the delayed onset burst release of therapeutic agents, the present invention also emphasizes abolishing the unstructured, poorly designed, and/or biologically incongruous geometry of conventional scaffolds by providing aligned scaffolding with parallel fibers or grooves. The aligned fibers or grooves can serve (i) as a substrate to guide and control natural endothelial cell deposition or (ii) as reservoirs for therapeutic agent(s) so that elution doesn't interfere with natural blood flow or cell deposition. The intravascular scaffold can be a highway to natural endothelization or a roadblock, depending upon the uniformity, alignment, and orientation of the constituent materials (i.e. fibers or grooves) of which it is composed.

Another company, Conor Medsystems, Inc. (acquired by Johnson & Johnson) directed its efforts to the controlled therapeutic agent delivery process. However, the therapeutic agent wells of the Conor CoStar™ stent took the form of dots rather than channels. These wells were neither longitudinally aligned nor continuous. Thus, the failure of the CoStar™ stent is suspected to be due, in part, to the inability of the spotted reservoir system to encourage structured endothelization.

These activities overlook the fact that even non-polymer coated therapeutic agentless stents, known as bare metal stents (BMS), cause thrombosis without antiplatelet treatment immediately post implantation and cause restenosis long-term even with antiplatelet treatment. Antiplatelet therapeutic agents are not necessarily also antirestenosis therapeutic agents and, regardless, they are not administered long-term following BMS implantation.

During stent placement, damage to mural tissue bordering the vessel lumen instigates an immune response. Popular traditional and current approaches to preventing restenosis characterize this immune response as something to be avoided. Current methods for avoiding the immune response that causes restenosis are directed at formulating more biocompatible stent coatings and therapeutic agents. The biocompatible coatings might mask the foreign nature of the stent or be more atraumatic to reduce the chance of irritating the lumen wall. Modern therapeutic agents aim to directly counteract the immune response by inhibiting it. These approaches and methods do not adequately address late stent thrombosis. When a biocompatible camouflage coating breaks down eventually the body will begin recognizing and reacting to the foreign nature of the stent and by that point in time all of the inhibiting therapeutic agent(s) have been exhausted. This can cause late stent/stage thrombosis.

The typical healing process of the body reacting to an implanted foreign body is for the body to encapsulate and otherwise wall off the foreign body. The present invention takes advantage of this principal, recognizes the beneficial value of a controlled immune response and provides a stent to work with the natural response rather than trying to avoid it by burying the stent with coatings and therapeutic agents to suppress it. The objective of the present invention is to provide a stent capable of eliminating both detrimental (uncontrolled) restenosis and thrombosis (both initially and at later stages, i.e. after six months of stent implantation). This avoids the current tradeoff that must be made between the two equally important goals ((i) no restenosis, (ii) no thrombosis) required by the choice between BMS and conventional DES.

When conventional BMS (i.e. without therapeutic agents or an aligned coating) are implanted, the new endothelium that develops is typically dysfunctional and does not effectively inhibit restenosis. This dysfunctional endothelium causes problems in the long term post-implantation in the form of uncontrolled restenosis. Thus, the practice of eluting antiproliferative therapeutic agents from stents to inhibit restenosis during the initial post-implantation period developed. The endothelium that develops on stents with unaligned surfaces or coatings is dysfunctional because the non-aligned struts do not merge well with the naturally aligned elongated endothelial cells (ECs) and proteins traversing a healthy blood vessel. It is easier for non-endothelial cells to form upon an unaligned, unstructured stent than it is for endothelial cells to integrate themselves. Therefore, the cells that grow to become the new lining are not true endothelial cells and that is at least part of the reason why the post-implantation in vivo “endothelial” layer formed on conventional (unaligned) stents is dysfunctional.

Some references disclose the “in vivo” adherence of endothelial cells to the surface of the stent (i.e. see U.S. Pat. No. 7,037,332 of Kutryk, et al.). The Kutryk (U.S. Pat. No. '332) patent discloses an antibody in a coating on a medical device that reacts with a surface antigen of natural endothelial cells to induce their adherence to the device. Kutryk relies upon a surface antigen rather than aligned fiber geometry to induce endothelization.

Some references, such as U.S. Pat. No. 6,855,366 by Smith, et al. (and assigned to the University of Akron) acknowledge some of the advantages of nitric oxide delivery using nanofibers. However, the Smith patent is limited to fibers of poly(ethylenimine). Further, Smith does not recognize: (i) the importance of aligning the fibers to facilitate functional endothelization, nor (ii) the possibility of using fibers as a coating to delay the onset of therapeutic agent release for therapeutic agents other than nitric oxide.

United States Patent Application No. (hereinafter US Pub. App.) 20080172124 (published Jul. 17, 2008) entitled “Multiple therapeutic agent-eluting coronary artery stent for percutaneous coronary artery intervention” (by Robert Bjork and presently unassigned) discloses a multi-layer stent that, like the present invention, is designed to address the problem of uncontrolled restenosis and to enhance endothelial in-growth. (See Abstract) It recognizes the need in the art for a solution capable of significantly reducing the incidence of thrombosis and restenosis “over an extended period of time.” However, the stent design and methods of functioning relied upon to resolve the restenosis problem and achieve positive effects (i.e. thrombosis prevention, neointimal hyperplasia inhibition, neovascularization suppression, endothelization facilitation, etc.) according to US Pub. App. '124 differ significantly from the design and methods taught in the present invention. For example, the present invention teaches controlling ECM development as one means for reducing problematic restenosis. One specific way to achieve this is through the elution of an ECM-suppressing therapeutic agent from the stent. US Pub. App. '124 does not acknowledge regulating or controlling ECM development. Instead it refers to the permanent affixation of an actual ECM molecule (rather than an ECM-suppressing therapeutic agent) on the stent or other implant. More specifically, the ECM molecule could be selected from “laminen, heparin, heparin sulfate proteoglycan (HSP), elastin, and fibronectin, chondroitin”. The ECM molecules are included to “enhance attachment and in-growth of normal endothelial cells into the stent lumen” rather than for any impact they may have on ECM development or other ECM molecules and components outside of the stent. There is no mention of the ECM suppressing therapeutic agents (i.e. fluoroquinolone, glucosamine, diethylcarbamazine, etc.) employed in the present invention.

Additionally, the publication is clearly directed at a coating that provides for an “immediate and sustained release of the anti-proliferative agent and the anti-inflammatory agent upon implantation”. A preferred embodiment of the present invention focuses on a coating that provides a delayed onset burst release of therapeutic agents. The publication teaches away from this by instructing an initial rapid release “within twenty-four hours of surgery” followed by a long-term “slower, steady delivery . . . over the next six to twelve months” ([0081]). Clearly, the therapeutic agent release profiles thought to be optimal differ between the publication and the present invention. The publication notes that a therapeutic agent combination preferably remains on the implant “between seven and thirty days” ([0043]). This teaches that all of the therapeutic agent can be gone in under twenty five days. In contrast, according to a preferred embodiment of the present invention, the therapeutic agent doesn't even begin eluting until twenty five days after implantation.

The chemical make-up and design of the stent coating (which may also be a therapeutic agent coating) disclosed in the publication also differs from the present invention in that the publication suggests using a “hydrophobic pharmaceutical agent” ([0071]) in a mostly hydrophobic coating (mass ratio of hydrophilic to hydrophobic polymers between 1:100 and 1:9). In contrast, the present invention teaches a hydrophilic active agent (i.e. fluoroquinolones) in a coating that is mostly of the opposite nature (hydrophobic) interspersed with hydrophilic pockets. Thus, in the present invention the mass ratio of the coating is likely to be dominated by material that is the opposite of the active agent with respect to its interaction with water (polar vs. non-polar, hydrophilic vs. hydrophobic).

U.S. Pat. No. 7,279,175 (“Stent coated with a sustained-release therapeutic agent delivery and method for use thereof” by Chen et al.) assigned to Psivida, Inc. of Watertown, Mass. covers a device and method for treating tissues. The device is a coated stent with a sustained-release therapeutic agent delivery system having a therapeutically beneficial advantage of reducing the incidence, recurrence, or both, of restenosis. U.S. Pat. No. '175 specifies a “coating comprising a polymer matrix having a low solubility protherapeutic agent dispersed therein”. It also discloses that the “low solubility protherapeutic agent is represented by the general formula A-L-B” wherein “A represents a therapeutic agent moiety having a therapeutically active form”; “L represents a covalent linker linking A and B to form a protherapeutic agent”; and “B represents a moiety which, when linked to A, results in the protherapeutic agent having a lower solubility than the therapeutically active form of A and is biologically or pharmacologically inert upon cleavage from the protherapeutic agent”. Thus, the claims of U.S. Pat. No. '175 are limited to coatings with low solubility therapeutic agent complexes in which the solubility of a sustained release therapeutic agent is reduced by a specific moiety that binds with the therapeutic agent. When therapeutic agents are provided upon and/or within the medical devices of the present invention special moieties are not required to bind with the therapeutic agents for reducing their solubility in order to control release. Rather, the present invention provides highly soluble stand-alone therapeutic agents protected in a matrix that will immediately demonstrate their high solubilities in the new surrounding environment that present itself when that matrix is penetrated and degrades.

U.S. Pat. No. '175 focuses on control in the form of stabilizing or prolonging release so that it doesn't happen all at once. The present invention focuses on control in the form of delaying release. In a preferred embodiment of the present invention once a threshold or trigger point is reached no control prevails as the therapeutic agent is allowed to flood the site quickly and completely. It appears that in U.S. Pat. No. '175 the only dramatic change is upon decoupling of the binding moiety from the therapeutic agent to transform the therapeutic agent into an active form. This transformation is part of therapeutic agent activation rather than release. In any case, comprehensively the system is controlled such that the drama of individual moiety-therapeutic agent complexes decoupling produces a persistent and steady supply of activated therapeutic agent (13:66-14:25).

In accordance with the present invention the biodegradable coating itself (rather than a specific moiety or binding agent within the coating) can act as a switch to determine time, intensity, and other characteristics of release of an antiproliferative, antirestenosis, and/or extracellular matrix suppressing therapeutic agent. The coating and/or matrix may prevent the initial elution of a therapeutic agent independently of the surrounding environment (dependent on time alone) based on a natural degradation time for the coating/matrix material. Alternatively, in an event-triggered approach the time of elution may depend on the environment (i.e. encapsulation) surrounding the coating/matrix that shields the therapeutic agent from elution. For example, in the event-triggered approach, one or more change in the environment of the coating/matrix that accompanies a desired amount of encapsulation is responsible for initiating the degradation of the coating/matrix. In one embodiment, an outer layer of the coating/matrix is the first to degrade in a time-dependent and/or event-dependent manner. Subsequently, an inner layer (including an arrangement of pockets in the outer layer) of the coating/matrix is exposed and itself breaks down immediately upon exposure to the restenotic tissue enabled by degradation of the outside layer/matrix. Delayed elution of the therapeutic agent from the shielding layers will initially (for 5-60 days post-implantation) allow natural immune response reactions to occur, creating a natural coating on the device and then, preferably releasing a therapeutic agent to inhibit the immune response thereby controlling restenosis. This method of the present invention thus emphasizes a burst, sudden, or pulsatile therapeutic agent release following a delayed or suspended onset. U.S. Pat. No. '175 emphasizes a “steady” (14:19-25) “sustained-release” and only baldly suggests a delayed release once (4:29-33) without teaching what the delay period should correspond to, if anything. The present invention teaches the relationship between the delay period, optimal tissue encapsulation, and biodegradable coating breakdown. U.S. Pat. No. '175 also neglects to teach anything about stent material (i.e. fiber) alignment/orientation or control of the extracellular matrix/proteoglycans. Although ciprofloxacin is disclosed as an antibiotic (14:31, 14:58, etc.) and as a base acceptable for use as a pharmaceutically active compound (17:45-55), any role it may play in ECM control is neither disclosed nor suggested. Other materials (i.e. glucosamine and diethylcarbamazine) taught in the present invention for ECM regulation are also undisclosed.

### BRIEF SUMMARY OF THE INVENTION

The present invention presents medical devices and methods for their operation such that the devices will be accepted by the body in the short-term and the long-term while preventing excessive build up of tissue from uncontrolled immune response. The present invention focuses primarily on controlling immune response by impacting smooth muscle cells and extracellular matrix (ECM) components. By controlling the body's immune response (i.e. as manifested in thrombosis, restenosis, and excessive ECM thickening), the invention disguises the stent with the body's own tissue in vivo. The tradeoff between short-term and long-term benefits and between the advantages of conventional bare metal stents (BMS) and contemporary therapeutic agent-eluting stents (DES) are avoided as the present invention combines the advantages of both stent types to provide immediate and enduring benefits.

The present invention teaches the value of letting natural immune response reactions occur during the first 5-60 days following implantation of a medical device and further teaches how to facilitate those reactions so that they encapsulate the device with aligned cells and do not become exacerbated to compromise blood flow through the vessel. According to the principles of the present invention an immune response is desirable because it can induce a biological reaction that results in aligned endothelialization covering the stent struts. Once the stent struts are smoothly covered, the chance of a more harmful immune response (late stent/stage thrombosis or LST) can be reduced or eliminated because the hemodynamic environment is not conductive to thrombosis or other problematic issues. No stent coating is more biocompatible than one made in vivo, from naturally synthesized biomaterials such as the tissue (i.e. aligned endothelial cells) generated by the body's own process of reacting to a foreign body (i.e. implant such as a stent). The creation of natural coatings in vivo avoids an aggravated immune response from the body reacting to foreign materials. Thus, when a natural tissue coating is provided in an optimal amount (thickness) and with optimal geometry (smooth and even) and one or more therapeutic agent is released preferentially or exclusively after formation of the coating the following negative consequences are avoided; inflammation, excessive vessel narrowing restenosis, clotting, excessive smooth muscle cell migration and proliferation, hyperplasia, and thrombosis.

After proper healing of a thin layer of the body's own tissue over the stent, the stent is configured to begin controlling subsequent restenosis and/or extracellular matrix development. If the extracellular matrix is left to develop unchecked it can produce flow compromising narrowing of the vessel. One means (but not the exclusive or only means) for controlling the proliferation of the extracellular matrix to suppress detrimental thickening is by eluting a therapeutic agent from the medical device that interacts with the components and proteoglycans that make-up the ECM. Likewise, the same or a different therapeutic agent may also be used to interact with components (i.e. smooth muscle cells or SMCs) and endothelial cells responsible for other aspects of restenosis. The timing of elution for one or more therapeutic agent or other non-therapeutic agent means for impacting natural tissue development can and should be delayed if that therapeutic agent or other non-therapeutic agent means also interferes with the desirable tissue development (i.e. aligned endothelialization) that creates the smooth, streamlined, coating that disguises the foreign character of the device. However, optionally, if the selected therapeutic agent or non-therapeutic agent means does not unacceptably interfere with positive results, one or more therapeutic agent may be eluted (or one or more means may be triggered) immediately upon implantation without a delay period. For example, in one scenario a first targeted substrate-specific therapeutic agent that interacts selectively with ECM components may be eluted immediately because it doesn't prevent positive endothelization while a second more general antiproliferative may be eluted after a substantial delay period because earlier elution would interfere with creation of the camouflage layer of aligned endothelial cells. Delaying the release of ECM suppressing therapeutic agent until ECM deposition begins makes the ECM suppression more effective. The present invention teaches the design and method for accomplishing this.

### BRIEF DESCRIPTION OF THE SEVERAL VIEWS OF THE DRAWINGS

FIG. 1 shows a restenosis cascade indicating at what point in time following the implantation of a stent various biological activities have occurred. The invention provides for elution of a restenosis or ECM suppressing therapeutic agent (which will bring such biological activities to a halt) anywhere from 5-60 days following stent implantation.

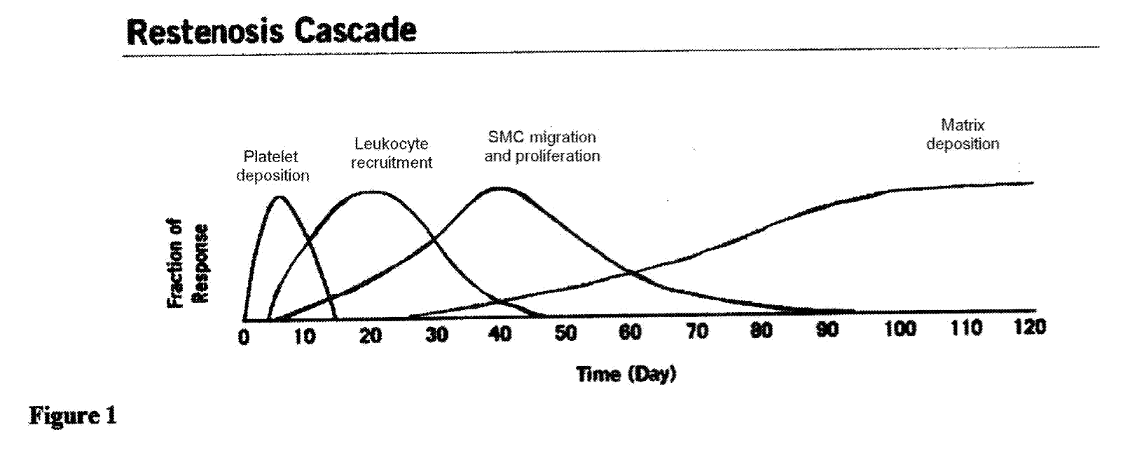

FIG. 2 shows the cumulative amount of therapeutic agent released from popular therapeutic agent eluting stents in the days following stent implantation as compared with the stent of the present invention. FIG. 2 demonstrates how the delay coating on the stent of the present invention suppresses therapeutic agent release until approximately 25 days after the stent is properly positioned in contrast to conventional DES that only slow the rate of release but provide immediate release. The Taxus® DES provides a steady slow release rather than a delayed burst or pulsed release.

FIG. 3 is a side cross-sectional view of the stent struts (zig-zag or sinusoidal in shape) and aligned fiber coating with the aligned fibers in a staggered pattern connecting adjacent struts. The struts provide radial columnar strength and support while the aligned fibers provide longitudinal flexibility.

FIG. 4 shows the initiation of therapeutic agent elution from a therapeutic agent matrix upon a stent strut after degradation of a delay coating.

FIG. 5 shows an ECM suppressing therapeutic agent matrix layer inside a first amphipathic (polar, partly hydrophilic) inner layer and a second amphipathic (weak polar, partly hydrophobic) outer barrier layer to create a delayed onset, sudden burst release of the therapeutic agent. Additional iterations of this therapeutic agent matrix-layering pattern may optionally be included to provide more than one burst release or a pulsatile release pattern with discrete release and recovery periods. This illustration assumes the selected therapeutic agent is at least slightly hydrophilic.

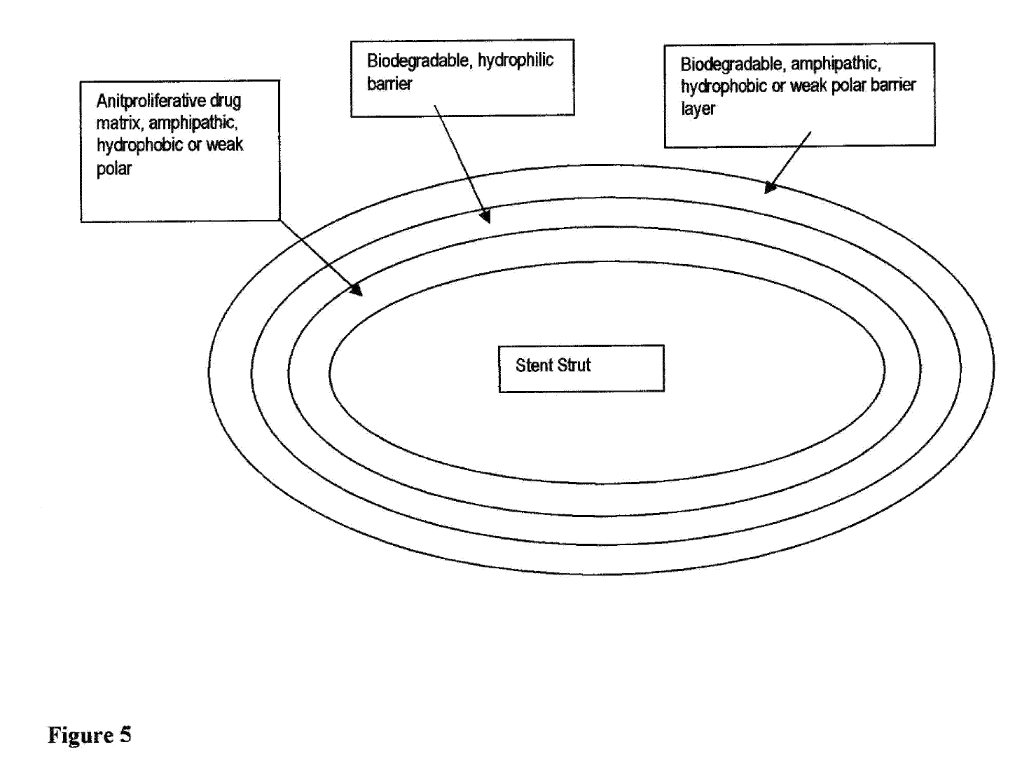

FIG. 6 shows a variation of the arrangement in FIG. 5 in which instead of first and second layer outside of the therapeutic agent matrix layer there is a single layer in which the “first layer” (similar in polarity to the therapeutic agent) has been transformed into pockets encapsulated in the second barrier layer (opposite in polarity to the therapeutic agent).

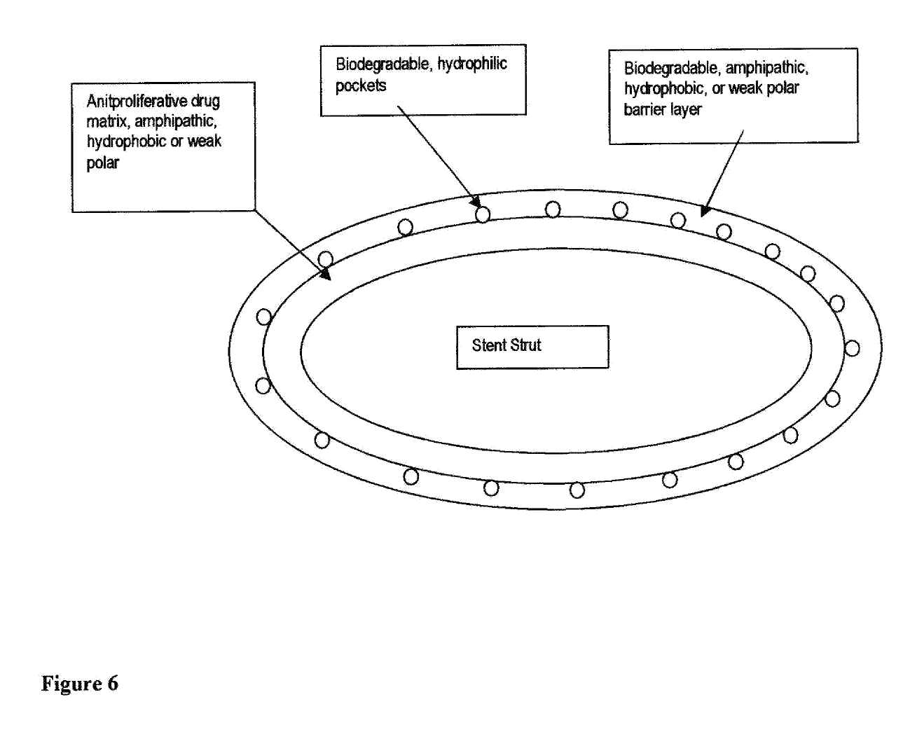

### DETAILED DESCRIPTION OF THE INVENTION

### I. Definitions

In the present invention the following terms are defined as follows:

“Antiproliferative” is used to refer to a substance that has the effect of inhibiting thickening, volume, or mass growth of tissue generally, including in the space surrounding an implant. The term includes “antirestenosis” and extracellular matrix-suppressing. The term may used in association with therapeutic agents and/or therapeutic agents and if not otherwise specified refers to both therapeutic agents and therapeutic agents and any other substance that have the given effect.
“Antirestenosis” is used to refer to a substance that has the effect of inhibiting thickening, volume, or mass growth of tissue that would otherwise restrict a passageway within a lumen or vessel, including tissue development on an implant, such as a stent, that would thereby decrease the inner diameter of a vessel in which the stent is positioned.
“Coating” is used to refer to a layer on an implant, such as a stent, that is comprised of material different than the struts and fibers of the stent and may or may not include a therapeutic agent incorporated therein.
“Drug” is used to refer to classical pharmaceutical therapeutic agents which typically are represented by complex chemical formulae. It includes brandname drugs, generic drugs, and any later-discovered compounds that may be found to have effects similar or superior to those of currently known drugs.
“Matrix” is used to refer to a structural framework of something. It may be used to describe the basic structure of a stent of a coating layer upon a stent. The matrix may or may not include a therapeutic agent incorporated therein.
“Therapeutic agent” is used to refer to any material, substance, compound, factor, chemical, biological composition, drug, etc. that has a beneficial effect. It includes both naturally occurring and synthetic materials. The beneficial or therapeutic effect includes but is not limited to: suppressing an ECM component, inhibiting restenosis, preventing clotting, preventing thrombosis, suppressing excessive proliferation or one or more tissue type, encouraging synthesis/migration/aligned deposition of endothelial cells, etc.

### II. Discussion

In the simplest form of the present invention, a biodegradable layer is designed to act as a switch to turn on the release of an ECM suppressing therapeutic agent (i.e. fluoroquinolone, glucosamine, diethylcarbamazine, etc.) once enough proliferation has occurred to encapsulate the stent strut or when ECM deposition begins. Exemplary fluoroquinolones include ciprofloxacin, levofloxacin, and moxifloxacin. This can be achieved by timing the switch to match the typical time (Encapsulation Development Time) for development of tissue encapsulation (timing approach) or to have the encapsulation event itself trigger the switch (event triggered approach).

Under the timing approach, a biodegradable layer can be coated on the therapeutic agent matrix that would degrade enough to allow therapeutic agent elution around 20 to 40 days, the typical time of tissue encapsulation of a stent strut. The layer could be configured to degrade in tissue and/or in blood. For the switch to be effective, it must effectively block ECM suppressing therapeutic agents from eluting for the duration of Encapsulation Development Time and then quickly turn on to fully elute a therapeutic agent to block proteoglycans (i.e. versican, decorin, biglycan), hyaluronan, inter-a-trypsin and/or collagen (types I and III) from being further synthesized and deposited. In this way significant ECM-related restenosis is prevented since proteoglycans and collagen are the dominant components of ECM. The ECM is responsible for the bulk of restenosis in the long term.

Since the typical ECM suppressing therapeutic agent (i.e. fluoroquinolone) is hydrophilic, a good solid barrier layer should be made of a hydrophobic or slightly hydrophobic substance to control the elution time and degradation time to better match the Encapsulation Development Time. This outer barrier layer of a more hydrophobic substance can be selected from polylactic acid (PLA), polyglycolic acid (PGA), a copolymer of PLA and PGA (PLGA), polycaprolactone (PCL), other biodegradable polyesters, polyamino acids, or other hydrophobic, biodegradable polymers.

Preferably, under the barrier layer and immediately adjacent to the therapeutic agent matrix layer another layer is provided that is instead slightly hydrophilic or closer in polarity to the therapeutic agent itself than the outer barrier layer. This middle layer is the key to the rapid, burst characteristic of therapeutic agent elution while the outer barrier layer is the key to the delayed onset characteristic of therapeutic agent elution.

As an alternative or as a complement to providing a separate layer beneath the barrier layer that is opposite in polarity to the barrier layer and closer in polarity to the therapeutic agent, the material used to form the therapeutic agent soluble material can be provided in pockets distributed throughout the barrier layer. By interspersing the barrier matrix with pockets of a hydrophilic substance (i.e. dextran, heparin) a switch effect for accelerated barrier layer degradation and therapeutic agent elution can be better achieved. Upon a threshold level of water penetration into the barrier matrix containing the pockets, the pockets increase in pressure to the point where they burst to destroy the barrier structure. The pockets act as isolated reservoirs or oases for hydrophilic physiologic and other fluids that the barrier layer's base material does not readily accept. Although the biodegradation of the barrier layer may be directed by other means such as the emergence of a restenotic environment in which the barrier layer dissolves, the incorporation of pockets allows additional options for fine-tuning the timing of barrier degradation by also making it indirectly susceptible to hydrophilic fluids and environments.

If the therapeutic agent happens to be hydrophobic rather than hydrophilic the polarities (hydrophobicity and hydrophilicity) of the respective matrices, layers, and/or pockets should be reversed. The bottom line is that the outermost barrier layer is to be opposite in polarity to the therapeutic agent and the inner layer(s) or pocket(s) that are closer to the therapeutic agent are closer in polarity to the therapeutic agent. However, preferably the therapeutic agent itself is contained in a matrix that is opposite in polarity for stabilization. The design is sandwich-like in configuration with the outer barrier and the therapeutic agent matrix analogized to pieces of bread between the unique opposite polarity inner layer or pockets analogized to the meat. The inner opposite polarity layer is the trigger to burst elution because the therapeutic agent easily desolves within it suddenly and completely.

Under the event triggered approach, there are several ways to trigger the switch to allow therapeutic agent elution to occur upon tissue encapsulation of the stent strut:
1. First, the coating covering the therapeutic agent matrix is designed to immediately break down to allow therapeutic agent elution upon tissue encapsulation. This can be achieved by coating the therapeutic agent matrix with a slightly to hydrophobic, biodegradable outer barrier layer that breaks down quickly upon the presence of a slightly to very hydrophobic environment such as provided by restenotic material. A thin layer of wax or a fatty substance exemplify the type of coating to be used. Specific examples of these include lipoprotein, collagen, polyamino acids, PLA, PLGA, and polycaprolactone,
2. Second, the ECM suppressing therapeutic agent can be bound to a molecule that inactivates the therapeutic agent until ECM factors (i.e. collagen, proteoglycans) are present.
3. Third, the switch can be turned on by other factors accompanying tissue encapsulation or extracellular matrix thickening including: hormones, enzymes, and/or peptides, etc.
4. Fourth, pressure can be used to induce release of the therapeutic agent, i.e. by housing the therapeutic agent within a semi-permeable membrane that bursts or by including pressure-building pockets within a barrier layer.
5. Fifth, pH changes can be used to induce release of the therapeutic agent if the material retaining (i.e. coating or serving as a matrix for) the therapeutic agent is sensitive to acids or bases and degrades (in tissue or in blood) upon being subjected to acidic or basic environments. In one embodiment, the therapeutic agent is coated with a slightly hydrophobic, acid-sensitive layer of PLGA. Tissue encapsulation of the stent strut can trap the PLGA and the acids produced from PLGA degradation. Subsequently, the concentration of acids is dramatically increased which leads to rapid degradation of the PLGA itself.

This event triggered approach offers a high degree of control of therapeutic agent elution and/or activation. The onset of therapeutic agent elution and/or the catalyst for therapeutic agent activation is particularized to occur independently and exclusively on the stent localities encapsulated by tissue while the elution is restrained and/or the therapeutic agent remains dormant and inactive on the stent localities that are still bare and unencapsulated. Encapsulation rates vary between procedures, individuals, and stent localities. Therefore, event-triggered therapeutic agent control provides an individualized approach for enhanced accuracy, safety and effectiveness.

It is preferred that the dosage of the anti-restenosis therapeutic agent is higher at the ends of the stent to compensate more aggressive restenosis at the ends of the stent.

In one embodiment, the present invention uses aligned nanofibers and/or aligned nanogrooves to form the stent coating to create an artificial functional endothelial layer that will attract the deposition of a natural endothelial layer. The natural endothelial layer is composed of aligned, elongated endothelial cells that will align themselves amongst the aligned fibers and deposit directly on the stent itself even when the aligned nanofiber coating is not loaded with any specifically reactive linking agents.

In contrast, the Kutryk patent (U.S. Pat. No. '332) only discloses amorphous carbon, fullerenes and hollow nanotubes (rather than aligned rod-like nanofibers) for the matrix material of a stent. Kutryk relies upon specific components, antibodies, to react with specific, known antigens in natural endothelial cells to create the first endothelial cell layer without any specific cell orientation. That is, the device, coating and methods of Kutryk “may stimulate the development of an endothelial cell layer with random cell orientation on the surface of the medical device” (see U.S. Pat. No. '332) but they do not themselves serve as an aligned functional endothelial cell layer.

The xenographic/xenogenic artificial functional endothelial layer of aligned fibers and/or aligned grooves may be composed of or seeded with synthetic materials, allogeneic materials (cells or clones from a second subject of the same species as the patient), and/or heterologous materials (cells or clones from a second subject not of the same species as the patient). In any case, the aligned geometry of the artificial functional layer paves the way for the growth of a natural functional layer of autologous endothelial cells produced in vivo that will encapsulate the stent struts and injured to tissue to a depth of 0.1 mm thereby masking its xenographic (foreign) nature to preclude an immune response that may cause thrombosis.

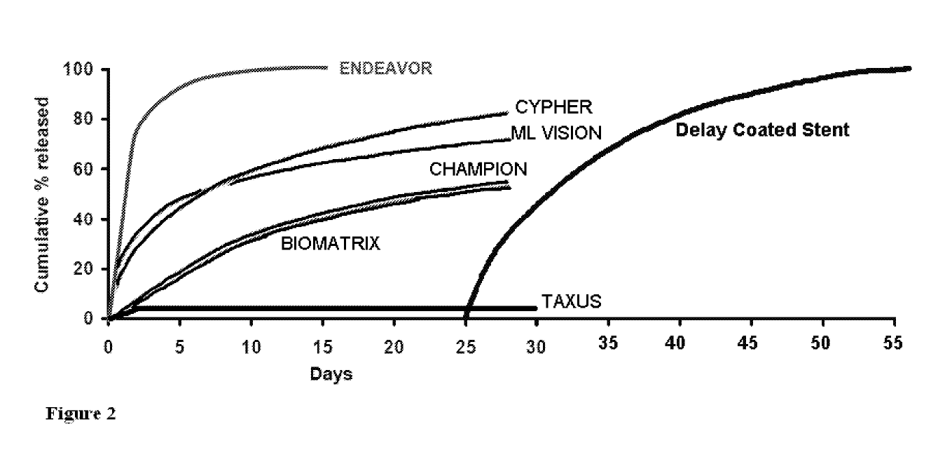

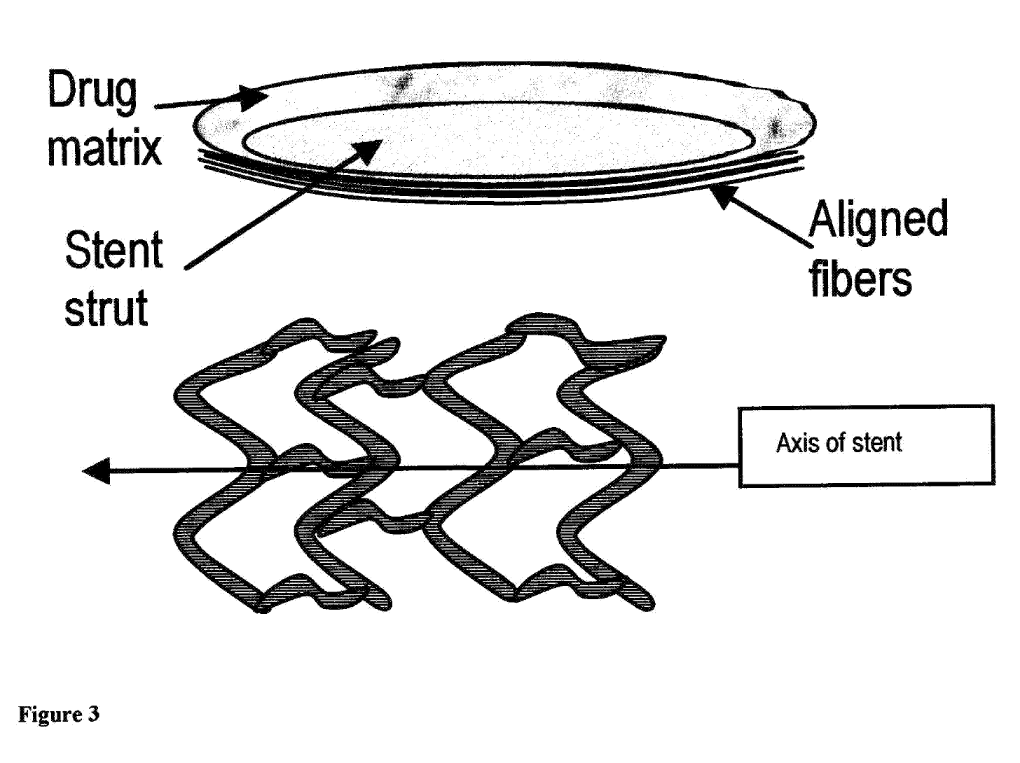

The present invention is a novel approach to solving the problem of LST without sacrificing the effectiveness of using restenosis suppressing therapeutic agents to avoid late stage restenosis and using ECM regulating therapeutic agents to reduce thickening of the ECM. This is done by depositing a biodegradable layer of aligned microfibers (AMF), aligned nanofibers (ANF), and/or aligned grooves (AG) on top of a DES as an effective means to delay the onset of release of one or more therapeutic agent (i.e. restenosis or ECM inhibitory therapeutic agents) as well as to facilitate endothelization (see FIG. 2 and FIG. 3). This way the patient benefits from two desired characteristics:
1. the safety of the BMS by having a smooth endothelium or neointima encapsulating the stent struts; and
2. the long term effectiveness of proven DES (such as Cypher and Taxus) by maintaining delivery of a local restenosis and/or ECM suppressing therapeutic agent from the stent but with a delayed onset.

The AMF/ANF/AG material may take the form of a coating, a matrix, or a stent body so long as its structure and orientation are such that it can both facilitate endothelization and also delay the onset of therapeutic agent release, if therapeutic agents are used. Preferably, the AMF/ANF/AG material lasts for 15-30 days before it is fully degraded to expose the therapeutic agent underneath. However, it may work by fully degrading anywhere between 5-60 days. The AMF/ANF/AG material is preferably made of PGA or a copolymer of PGA-PLA. These are proven compounds used on DES as well as biodegradable sutures and are well documented for their compatibility with blood. PGA and PGA-PLA are especially well suited to degrade within 15-30 days. The delay time before onset of release of the ECM suppressing therapeutic agent (i.e. fluoroquinolone, glucosamine, diethylcarbamazine, etc.) is equal to the time it takes the AMF/ANF/AG material to fully degrade. This delay time is controlled by the exact chemical compounds used to create the coating and also the coating thickness. For example, since 50% PLA:50% PGA degrades more quickly than a 75% PLA:25% PGA mix, to obtain the same therapeutic agent release onset delay a thicker layer of 50% PLA:50% PGA would be used than if a 75% PLA:25% PGA mix were used. The AMF/ANF/AG material is preferably between 0.1 micron and 20 microns thick.

Alternatively, instead of PGA and/or PLA, the AMF/ANF/AG material can also preferably be made of poly(ethylene glycol) (PEG), also known as poly(ethylene oxide) (PEO) or polyoxyethylene (POE). Caprolactone (CPL) can also be used. CPL and PEG are elastomeric materials and if the AMF/ANF/AG medical device has elastomeric properties it will better conform to the natural shape of the lumen in which it is inserted or implanted. Elastomeric materials are better able to close gaps between a stent wall and a lumen wall. Avoiding incomplete apposition of the stent struts against the lumen wall reduces the formation of stagnant pockets in which a thrombus is more likely to develop. Metallic stent struts are typically stiff and cannot conform well to the lumen when the lumen is not smooth and uniform, as is often the case. However, an elastomeric coating upon non-elastomeric stent struts ameliorates this problem by flexing, bending, expanding, and contracting to occupy the differential spaces created by the nonconformity between the lumen wall and the stent struts. Alternatively, if the stent struts themselves are made of AMF/ANF/AG elastomeric materials they can directly model the irregular surface patterns of anatomic lumens.

The AMF/ANF/AG material can also be made out of biological molecules (biomolecules) such as collagen, fibrin, or fibrinogen. Various other substances that can be used to form the AMF/ANF/AG material are: phosphorylcholine, nitric oxide, high density lipoprotein, polyzene-F, PTFE polyetherester, hydroxyapatite, polyhydroxy-butyrate, polycaprolactone, polyanhydride, poly-ortho ester, polyiminocarbonates, polyamino acids, and polyvinyl alcohol.

Irrespective of the chemical components used to form the AMF/ANF/AG material, when used as a delay coating the AMF/ANF/AG material is preferably negatively charged and also preferably has a nitric oxide functional group. Thus, as the fibers degrade, nitric oxide is released. Within the bloodstream of the lumen occupied by the stent, the nitric oxide serves to further inhibit restenosis by preventing platelet aggregation and macrophage/leukocyte infiltration, reducing smooth muscle cell proliferation, and decreasing inflammation generally while aiding the healing process. An aligned coating with a nitric oxide group (ANO) on a stent (or other intravascular medical device) forms an artificial endothelium layer due to the smooth, streamlined surface the aligned fibers/grooves provide coupled with the ability of nitric oxide to prevent aberrations on this smooth surface as the fibers degrade.

The present invention recognizes the use of any biocompatible materials that can be formed into aligned nanofibers, aligned microfibers, or aligned grooves for the AMF/ANF/AG material used to form a stent, a coating, or a matrix for therapeutic agent(s). The present invention also recognizes the ability to use the AMF/ANF/AG material in conjunction with other coatings, layers, matrices, pores, channels, reservoirs, etc. to delay onset of the release of any therapeutic agent and/or to encourage structured (i.e. aligned) endothelization.

The present invention also teaches the criticality of matching the time period of delay prior to therapeutic agent release with the time it takes for the AMF/ANF/AG stent surface to become covered (i.e. encapsulated) by endothelization to a depth of approximately 0.1 mm. The artificial functional endothelium layer itself is a very thin (i.e. only one or a few cells thick). A thin layer does not burden the stent with unnecessary volume (i.e. on the periphery of a cross-section) that could make insertion and adjustment within the lumen more difficult. A thin layer also does not significantly reduce the inner diameter of the stent's lumen and therefore does not interfere with hemodynamics or obstruct blood supply to a treated area.

When the stent is not formed of a material (i.e. such as an elastomeric aligned material) that enables it to conform to the shape of a lumen surface, a thrombus is more likely to develop causing a localized inflammatory reaction. Also, when the stent doesn't conform well to the shape of a lumen, the process of restenosis cannot be effectively controlled. Although systematic therapeutic agents administered with BMS and therapeutic agents supplied by DES can slow or modulate the rate of ineffective restenosis they are not typically used to encourage a moderate amount of beneficial restenosis. Any restenosis that does occur in a vessel having an uneven surface with stent struts that inadequately conform to the natural cell and protein structure (and/or shape) of the vessel is likely to be uncontrollable and problematic. Smooth muscle cell migration and proliferation is likely to form the first tissue layer over the stent struts. In contrast, the present invention provides a pre-formed artificial functional endothelial layer to provoke a first in vivo layer of natural endothelial cell growth.

According to the present invention, an aligned (i.e. AMF/ANF/AG/ANO) coating on the luminal surface aligns both the blood flow and the growth of natural endothelial cell layers in a uniform, optimal direction (i.e. longitudinally along the central axis of the lumen). An aligned inner coating accelerates and optimizes blood flow for better drainage and support. Normal blood flow around the stent flushes out immune response agents and toxins, as they are produced, to accelerate drainage and healing. Normal blood flow also feeds the developing, natural endothelial cell layer above the artificial functional endothelial stent coating with nutrients.

Once the natural endothelial cell layer has developed to a sufficient extent (i.e. a depth of approximately 0.1 mm) and moderate amounts of beneficial (i.e. aligned) restenosis have been permitted to occur, the result is a camouflaged stent buried within normal, healthy tissue. No foreign materials are detectable by the blood and so the blood related immune response and inflammation are inhibited, thereby greatly reducing the risk of thrombosis. As therapeutic agents begin to be eluted from DES upon degradation of the aligned coating, the beneficial, controlled restenosis process (“encapsulation”) comes to a halt. The stent remains stably buried but the thickness of the luminal walls stops increasing to avoid reclosure. The therapeutic agents are powerful enough to prevent additional encapsulation but cannot undo the beneficial, stent-sealing, encapsulation that has already occurred.

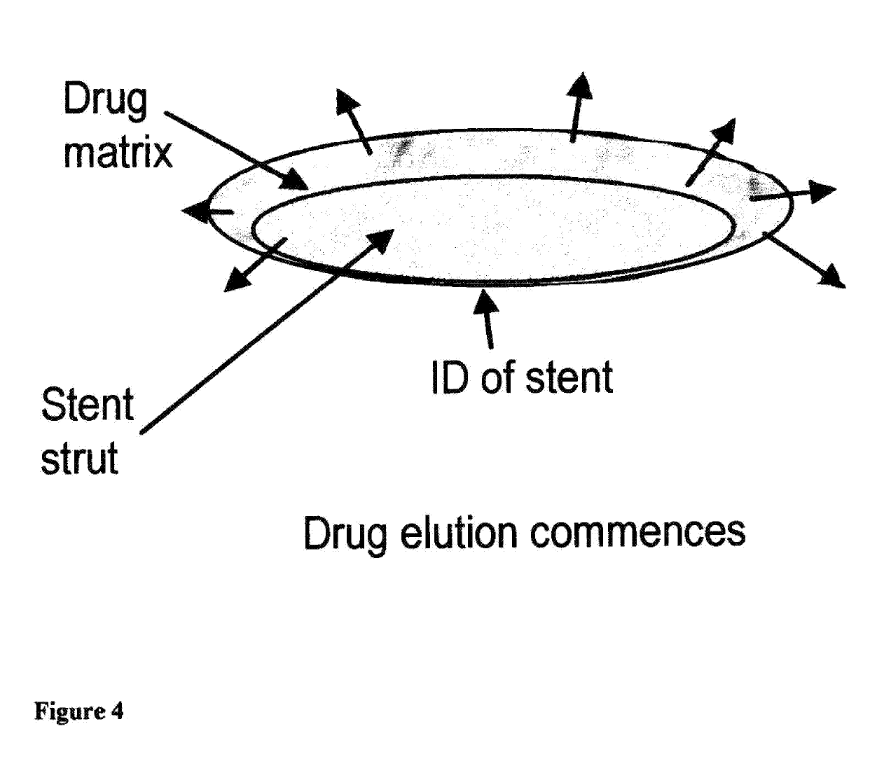

Elution of the therapeutic ECM suppressing therapeutic agent will arrest the proliferation of neointima (protein deposition) (see FIG. 4). Due to the delay in the onset of therapeutic agent release, by the time the therapeutic agents are released all the stent struts are encapsulated with endothelium and/or smooth muscle. Therefore, higher dosages of therapeutic agents, faster elution rates, and/or more aggressive therapeutic agents can be used to ensure maximum effectiveness in preventing restenosis and inhibiting excessive ECM thickening in the long term without fear of LST from an immune reaction. Once the stent struts are smoothly buried beneath a thin natural tissue layer thrombosis is unlikely.

Optionally, the stent may have semi-permeable cross-sectional side walls extending through the surface area of the cross section on each end adjacent to a target site to be treated with an eluted therapeutic agent. The side walls would serve as barriers to the therapeutic agent to concentrate it at the target site and avoid the negative effects of systematic therapeutic agent distribution. Such sidewalls would also conserve the therapeutic agent to be maintained where it is needed most to allow less total therapeutic agent within the stent to be equally effective by reducing the washout effect. Reducing the total therapeutic agent stored in the state (while maintaining effectiveness) is beneficial because then the stent walls can be thinner and it is also less expensive. The semi-permeable nature of the side walls allows them to permit the influx of important nutrients needed at the constricted vessel site and to permit the outflux of waste thus preserving hemodynamics. The cross-sectional side walls would dissolve naturally in time to correspond with the termination of the desired therapeutic agent treatment period.

Optionally, the stent may include radioopaque substances in one or more of the materials of which it is formed or in one or more coatings. An array of different, distinguishable radioopaque substances may also be used in each layer or coating. These substances would enable a physician to externally observe the placement, progress, and improvement of the stenting procedure without causing the patient discomfort from an internal inspection and without risking displacing the stent during an internal (i.e. endoscopic) inspection.

Another approach to avoiding LST while still controlling restenosis is by accelerating the endothelization of the stent through aligned scaffolding without the antirestenosis therapeutic agent. The bare stent can be made of (at least in part) or coated with elongated AMF/ANF/AG/ANO aligned with the direction of blood flow (i.e. long axis of fibers parallel to the direction of blood flow). Endothelial cells (ECs) are themselves elongated and tend to also be aligned with the direction of blood flow. By aligning the fibers with the preferred alignment of ECs, the deposition of ECs over the stent (including but not limited to the stent struts) is accelerated (aligned scaffolding). The presence of ECs tends to arrest the restenosis process (smooth muscle proliferation). The AMF/ANF/AG/ANO are preferably laid down on the inner diameter (ID) of the stent (see FIG. 3). The outer diameter (OD) or abluminal surface of the stent is typically embedded in or aligned against the luminal surface of the vessel so that the longitudinal alignment of the fibers here is not as important as for the inner diameter or luminal surface of the stent.

The stent struts are typically 50 to 100 microns wide. The fibers are preferably 0.5 to 10 microns wide. Therefore, regardless of the stent strut orientation, the fibers can have an aspect ratio of 5 or greater. By having an aspect ratio greater than 2, the fibers can provide effective longitudinally aligned scaffolding for ECs to grow on.

The AMF/ANF/AG/ANO coating or surface can be impregnated or coated with antiplatelet or anticoagulant therapeutic agents such as heparin, ticlopidine, chlopidrel, enoxaparin, dalteparin, hirudin, dextran, bivalirudin, argatroban, danparoid, Tissue Factor Pathway Inhibitor (TFPI), GPVI antagonists, antagonists to the platelet adhesion receptor (GP1b-V-IX), antagonists to the platelet aggregation receptor (GPIIb-IIIa) or any combination of the aforementioned agents.

The AMF/ANF/AG/ANO material can also be impregnated with endothelization promoting substances such as vascular endothelial growth factor (VEGF), angiopoietin-1, antibodies to CD34 receptors, and/or hirudin, dextran.

The coating can be applied to the inner diameter (ID) of the stent in the form of longitudinally aligned microfibers, nanofibers, grooves, or nitric oxide carrying elements by several modified processes of electrospinning:

1A. Aligned Nanofibers on stent struts only: A dispensing syringe is loaded with a solution of the fiber material and is charged (i.e. positive) with a high voltage (>1 kV) to charge the solution. The stent is either grounded or charged by applying the opposite voltage (i.e. negative). The outer diameter (OD) of the stent is covered with a polar or conductive tube that sticks to the fiber material well. For example, if PGA or PLA are used as the polymer solution from which the fiber material is formed, polyethylene terephthalate (PET) is heat shrunk on the OD of the stent. The stent is held by a grounded or charged (i.e. negative) collet on the OD of one end. The dispensing syringe needle with a 90 degrees bend (or side hole) at the tip is inserted inside the ID of the stent from the open end of the stent. The charged solution is dispensed from the needle tip onto the stent ID as longitudinally aligned micro/nanofibers/grooves/nitric-oxide carrying elements as the syringe tip is moved back and forth longitudinally. As the syringe tip completes one pass from one end to the other, the collet is indexed (turned incrementally) to lay down the adjacent fiber. This process continues until the whole stent ID is covered with aligned fibers, grooves or elements. Once the coating is finished, the cover (i.e. polar or conductive tube such as PET) on the OD can be peeled off to clear the stent openings of fibers.
1B. Aligned Nanofibers covering all stent: A dispensing syringe is loaded with a solution of the fiber material and is charged (i.e. positive) with a high voltage (>1 kV) to charge the solution. The stent is either grounded or charged by applying the opposite voltage (i.e. negative). The stent is held by a grounded or charged (i.e. negative) collet on the OD of one end. The dispensing syringe needle with a 90 degrees bend (or side hole) at the tip is inserted inside the ID of the stent from the open end of the stent. The charged solution is dispensed from the needle tip onto the stent ID as longitudinally aligned micro/nanofibers/grooves/nitric-oxide carrying elements as the syringe tip is moved back and forth longitudinally. As the syringe tip completes one pass from one end to the other, the collet is indexed (turned incrementally) to lay down the adjacent fiber. This process continues until the whole stent ID is covered with aligned fibers, grooves or elements.
2. The highly charged (i.e. +10 kV) syringe as described above is fixed longitudinally. The stent is grounded. A ring of opposite charge (i.e. −10 kV) is placed near the stent. The dispensing syringe is pulsed by pulsing syringe pressure, a needle valve, or charging to completely dispense one aligned fiber. The stent is then rotationally indexed for the next pulsed dispensing.
3. A hollow ring containing the solution of fiber material has series of micro/nano-holes on the end for dispensing parallel fibers arranged in a diameter close to the diameter of the stent. The ring is highly charged (i.e. +10 kV) to charge the fiber material in solution. The stent is grounded. A ring close to the diameter of the stent is charged with an opposite charge (i.e. −10 kV) on the opposite end of the stent. This charged state will cause the solution which forms the fibers to eject from the holes in parallel, longitudinally towards the oppositely charged ring while simultaneously adhering to the stent along the path from one ring to another.

In another embodiment, the inner surface of the stent strut can have micro/nano-grooves etched on it longitudinally (parallel to axis of stent). ECs will tend to grow into these grooves. The grooves are preferably 1 to 10 microns wide. In the same manner, the grooves can also be ridges or channels. The longitudinally aligned micro/nano-grooves may also be used as reservoirs or longitudinal wells for storing therapeutic agents within the aligned fiber layers for controlled or multi-phase elution.

These AMF/ANF/AG/ANO stents are particularly advantageous when applied to intravascular bifurcations or vessels with one or more corollary branch adjacent to a main lumen. Bifurcated vessels tend to have much higher rates of restenosis with both conventional BMS and DES than do non-bifurcated vessels.

The present invention controls tissue encapsulation of the stent and of injured tissue in at least three ways: biologically, geometrically, and chronologically.

Biologically, aligned nano/microfibers with or without aligned nano/microgrooves therein (or alternatively, aligned grooves formed within a non-fibrous material) facilitate functional endothelization by encouraging a uniform orientation in any cell growth that occurs (whether of true endothelial cells or artificial endothelial cells). The polymers or other materials chosen for the construction of the nano/microfibers or nano/microgrooves must be biocompatible to permit the natural flow of blood and other bodily fluids through the lumen adjacent the stent's inner surface without elicitation of an immune response or thrombosis. The materials used to form the fibers or the material within which the grooves are etched can be synthetic or naturally derived. Suitable materials include: biodegradable materials such as polyglycolic acid (PGA), polylactic acid (PLA), copolymer of PLA and PGA (PLGA), hydroxyapatite (HA), polyetherester, polyhydroxybutyrate, polyvalerate, polycaprolactone, polyanhydride, poly-ortho ester, polyiminocarbonates, polyamino acids, polyethylene glycol, polyethylene oxide, and polyvinyl alcohol; non biodegradable polymers such as fluoropolymer like Polytetrafluoroethylene (PTFE), polyzene-F, polycarbonate, carbon fiber, nylon, polyimide, Polyether ether ketone, polymethylmethacrylate, polybutylmethacrylate, polyethylene, polyolefin, silicone, and polyester; biological substances such as high density lipoprotein, collagen, fibrin, phosphorylcholine (PC), gelatin, dextran, or fibrinogen.

Geometrically, the invention is designed to only allow 0.1 mm thickness of encapsulation (of stent struts or the entire stent body and of injured tissue) before the therapeutic agent elution process begins to inhibit further encapsulation. Another aspect of geometric control is the alignment of fibers/grooves and all growth thereupon whether it be endothelial cells, smooth muscle cells, proteins, matrix fibers, or collagen fibers. Due to the structure supplied by the fibers/grooves, all subsequent in vivo growth, migration, and/or proliferation is necessarily aligned to correspond to the template set by the fibers/grooves. Aligned growth does not interfere with blood flow. Further, even if the initial natural layers of biologically derived materials deposited are not the ideal materials (i.e. smooth muscle cells instead of endothelial cells), as long as they are aligned they are suspected not to impede the deposition of the optimal materials when they come along.

Chronologically, the invention assures that the complete degradation of the polymer (or other material) layer serving as a delay coat for the antiproliferative therapeutic agent corresponds to the time when an optimal amount (i.e. 0.1 mm thickness) of encapsulation has occurred because that point in time also marks the onset of elution of the antiproliferative therapeutic agent which will suppress further thickening of tissue encapsulation. Temporal control over the elution of the antiproliferative and/or other therapeutic agents may also be achieved by an external activation means that signals for the aligned therapeutic agent reservoirs to begin elution. The external activation means may be electromagnetic radiation, infrared light, microwave radiation, x-ray radiation, etc. This type of external activation means would provide very precise control of the onset of therapeutic agent elution. Since the rate of encapsulation will vary from individual to individual and from procedure to procedure depending upon a multitude of factors, a pre-elution assessment (i.e. imaging for endothelial cell markers) of the extent of encapsulation can precede initiation of the external activation means to ensure elution does not begin prematurely.

The materials and dimensions described here are not meant to be limiting. The general concept can be extended to other specific embodiments or ranges.

From the above description of the invention, those skilled in the art will perceive improvements, changes and modifications. Such improvements, changes and modifications within the skill of the art are regarded as covered by the appended claims directly or as equivalents.

What is claimed is:

1. A medical device, comprising: a coating having a composition configured to (i) allow formation of a protective tissue over an implanted device, and (ii) control development of extracellular matrix near the implanted device.

2. The coating of claim 1, wherein the coating comprises a therapeutic agent configured to suppress thickening of the extracellular matrix (ECM).

3. The coating of claim 1, wherein the coating comprises a therapeutic agent configured to suppress thickening of the extracellular matrix (ECM) and a therapeutic agent configured to suppress smooth muscle proliferation.

4. The coating of claim 3, wherein a second therapeutic agent, different than the therapeutic agent configured to suppress thickening of the extracellular matrix (ECM), is provided to suppress smooth muscle proliferation.

5. The coating of claim 2, comprising a biodegradable, slightly hydrophobic barrier adjacent to one or more storage site for the therapeutic agent.

6. The coating of claim 5, wherein the barrier contains pockets of a hydrophilic substance.

7. The coating of claim 5, wherein the slightly hydrophobic barrier comprises at least one element selected from the group consisting of: polylactide, polylactic acid, polyglycolide, polyglycolic acid, polylactide-polyglycolide, polycaprolactone, polyamino acid and any copolymer thereof.

8. The coating of claim 2, wherein the therapeutic agent comprises at least one element selected from the group consisting of: fluoroquinolones (including ciprofloxacin, levofloxacin, and moxifloxacin), glucosamine, and diethylcarbamazine.

9. The coating of claim 3, wherein the therapeutic agent configured to suppress thickening of the extracellular matrix (ECM) is selected from the group consisting of fluoroquinolones, glucosamine, and diethylcarbamazine; and the therapeutic agent configured to suppress smooth muscle proliferation is selected from the group consisting of Paclitaxel, Rapamycin, Everolimus, Biolimus, Zotarolimus, Tacrolimus, fibroblast growth factor (bFGF), antisense dexamethasone, angiopeptin, Batimistat™, Translast™, Halofuginon™, acetylsalicylic acid, Tranilast™, estradiol, Hirudin, and any analog(s) or derivative(s) of the aforementioned therapeutic agents.

10. The coating of claim 6, wherein the hydrophilic pockets within the hydrophobic barrier comprise at least one element selected from the group consisting of: dextran, an ECM suppressing therapeutic agent, polyvinyl alcohol, polyethylene glycol (PEG, also known as poly(ethylene oxide) (PEO) or polyoxyethylene (POE)), gelatin, pullulan, heparin, hirudin, ticlopidine, chlopidogrel, a salt, and an anticoagulant.

11. The coating as claim 2, wherein the therapeutic agent configured to suppress thickening of the extracellular matrix (ECM) is contained in one or more hydrophilic pockets within a biodegradable polymer matrix that is more hydrophobic than the hydrophilic pockets.

12. The coating of claim 5, wherein the hydrophobic barrier repels the therapeutic agent.

13. The coating of claim 6, wherein the hydrophilic pockets consists of dextran and the hydrophobic barrier consists of 50-75% polylactic acid and 25-50% polyglycolic acid.

14. The coating of claim 1, further comprising a second coating wherein the second coating is a protective coating.

15. The coating of claim 1, wherein the coating is biodegradable, bioabsorbable, or bioerodable.

16. The coating of claim 1, further comprising an anti-thrombogenic substance.

17. The coating of claim 16, wherein the therapeutic agent for reducing clotting is selected from the group consisting of: heparin, ticlopidine, chlopidrel, enoxaparin, dalteparin, hirudin, dextran, bivalirudin, argatroban, danparoid, TFPI, GPVI antagonists, antagonists to the platelet adhesion receptor (GP1b-V-IX), antagonists to the platelet aggregation receptor (GPIIb-IIIa), and any combination of the aforementioned agents.

18. The coating of claim 2, wherein onset of elution of the therapeutic agent for suppressing thickening of the extracellular matrix is delayed to occur 14 days to 90 days after implantation of the medical device.

19. The coating of claim 2, wherein an amount of time for delaying onset of elution of the ECM suppressing drug corresponds to:
a. an amount of time it takes for at least one therapeutic agent-containing or therapeutic agent-covering layer to degrade; and
b. an amount of time it takes for most of the medical device to become covered by a thin layer of cells produced by endothelization and/or restenosis.

20. The coating of claim 2, wherein the medical device is a stent having struts on its luminal surface and further comprising more than one layer, wherein all layers collectively form the coating; wherein the coating layers are arranged from the stent struts to an outermost surface of the stent in the following order:
(i) a primer layer;
(ii) a layer comprising an antiproliferative therapeutic agent; and
(iii) a layer for delaying an onset of release of the antiproliferative therapeutic agent.
